(19) United States
(12) Patent Application Publication
(10) Pub. No.: US 2018/0070154 A1
(43) Pub. Date: Mar. 8, 2018

(54) MOTION DETECTING APPARATUS AND MOTION DETECTING SYSTEM

(71) Applicant: SEIKO EPSON CORPORATION, Tokyo (JP)

(72) Inventor: Ken WATANABE, Matsumoto-shi (JP)

(73) Assignee: SEIKO EPSON CORPORATION, Tokyo (JP)

(21) Appl. No.: 15/689,740

(22) Filed: Aug. 29, 2017

(30) Foreign Application Priority Data
Sep. 6, 2016 (JP) 2016-173420

(51) Int. Cl. H04Q 9/00; H01Q 1/27; A61B 5/11; A61B 5/00

(52) U.S. Cl. CPC H04Q 9/00; H01Q 1/273; A61B 5/11; G01P 15/02; A61B 5/74; A61B 5/6823; H04Q 2209/43; A61B 5/6824

(57) ABSTRACT

A motion detecting apparatus includes an inertial sensor and an antenna via which a signal containing information based on data outputted from the inertial sensor is transmitted to a notification apparatus attached to a user, and when the motion detecting apparatus is attached to the user, at least part of the antenna is located on a ground side.

### BACKGROUND

### 1. Technical Field

The present invention relates to a motion detecting apparatus and a motion detecting system.

### 2. Related Art

In recent years, a motion analyzing system that analyzes motion is required in a variety of fields. JP-A-2013-233342, for example, describes that an activity quantity meter including a display section is attached to the waist of a person's body by sandwiching a belt, a waist band of trousers, or any other object between an attachment member and a main body of the activity quantity meter. Further, JP-T-2012-524640 describes an electronic module (wristwatch) that wirelessly communicates with a sensor (heart rate monitor, for example) worn on a user, is worn around the user's wrist, and has a display function.

In the motion analyzing system (motion detecting system) described above, however, for example, in a case where the wrist around which the electronic module is worn is in a position in the user's traveling direction (in front of user), the user's body is present between a motion detecting apparatus including the sensor and worn on the waist and the electronic module, so that the wireless communication between the motion detecting apparatus and the electronic module is unstable in some cases.

### SUMMARY

An advantage of some aspects of the invention is to provide a motion detecting apparatus capable of stably performing wireless communication. Another advantage of some aspects of the invention is to provide a motion detecting system including the motion detecting apparatus.

The invention can be implemented as the following forms or application examples.

### Application Example 1

A motion detecting apparatus according to this application example includes an inertial sensor, and an antenna via which data outputted from the inertial sensor is transmitted to a notification apparatus attached to a user, and when the motion detecting apparatus is attached to the user, at least part of the antenna is located on a ground side.

In the thus configured motion detecting apparatus, a signal transmitted via the antenna can be more reliably reflected off the ground, whereby the motion detecting apparatus can stably perform the wireless communication.

### Application Example 2

The motion detecting apparatus according to the application example may further include an electronic substrate connected to the antenna.

The thus configured motion detecting apparatus can stably perform the wireless communication.

### Application Example 3

In the motion detecting apparatus according to the application example, the inertial sensor and an electronic part may be mounted on the electronic substrate, and in a plan view viewed in a direction that intersects a first surface that is worn on the user, at least part of the antenna may be so located as not to overlap with the electronic part or the inertial sensor.

The thus configured motion detecting apparatus allows the distance between the antenna and the inertial sensor and the distances between the antenna and the electronic part to be increased. The motion detecting apparatus can therefore suppress entry of noise into signals transmitted by the inertial sensor and the electronic part via the antenna.

### Application Example 4

The motion detecting apparatus according to the application example may further include a box-shaped enclosure that accommodates a battery, and when the motion detecting apparatus is attached to the user, the battery may be located on a side opposite a side facing the user's site to which the motion detecting apparatus is attached.

In the thus configured motion detecting apparatus, the distance between the user and the battery can be increased. The influence of heat that the user receives from the battery can therefore be reduced.

### Application Example 5

In the motion detecting apparatus according to the application example, the electronic substrate may be disposed between the first surface and the battery.

In the thus configured motion detecting apparatus, the electronic substrate allows the user to unlikely to receive the influence of heat radiated from the battery.

### Application Example 6

The motion detecting apparatus according to the application example, may further include a terminal for external connection, and when the motion detecting apparatus is attached to the user, the terminal may be located on a ground-side side surface out of side surfaces of the enclosure.

The thus configured motion detecting apparatus can suppress entry of sweat of the user, rainwater, and other objects into the motion detecting apparatus via the terminal. As a result, in the motion detecting apparatus, rust and other undesirable phenomena due to sweat of the user, rainwater, and other objects is unlikely to occur.

### Application Example 7

The motion detecting apparatus according to the application example may be worn on the user's waist.

The thus configured motion detecting apparatus can stably perform the wireless communication.

### Application Example 8

A motion detecting system according to this application example includes the motion detecting apparatus according to the application example, and a notification apparatus.

The thus configured motion detecting system can stably perform wireless communication between the motion detecting apparatus and the notification apparatus.

### Application Example 9

In the motion detecting system according to the application example, the notification apparatus may be worn on the user's arm.

In the thus configured motion detecting system, for example, even when the motion detecting apparatus is worn on the user's waist, and the notification apparatus is worn on the user's arm, so that the user's body is present between the motion detecting apparatus and the notification apparatus, a signal transmitted from the antenna is radiated toward the ground, reflected off the ground, and received by the notification apparatus. The motion detecting system can therefore stably perform the wireless communication between the motion detecting apparatus and the notification apparatus.

### BRIEF DESCRIPTION OF THE DRAWINGS

The invention will be described with reference to the accompanying drawings, wherein like numbers reference like elements.

FIG. 1 is a perspective view diagrammatically showing a motion detecting apparatus according to an embodiment of the invention.

FIG. 2 is a cross-sectional view diagrammatically showing the motion detecting apparatus according to the present embodiment.

FIG. 3 is a perspective view diagrammatically showing a USB terminal of the motion detecting apparatus according to the present embodiment.

FIG. 4 is a perspective view diagrammatically showing a USB terminal of the motion detecting apparatus according to the present embodiment.

FIG. 5 describes connection between the motion detecting apparatus according to the present embodiment and a personal computer.

FIG. 6 is a perspective view diagrammatically showing the motion detecting apparatus according to the present embodiment.

FIG. 7 describes connection between the motion detecting apparatus according to the present embodiment and the personal computer.

FIG. 8 is a perspective view diagrammatically showing a motion detecting apparatus according to a first variation of the present embodiment.

FIG. 9 is a cross-sectional view diagrammatically showing the motion detecting apparatus according to the first variation of the present embodiment.

FIG. 10 is a perspective view diagrammatically showing a motion detecting apparatus according to a second variation of the present embodiment.

FIG. 11 is a perspective view diagrammatically showing a motion detecting apparatus according to a third variation of the present embodiment.

FIG. 12 is a cross-sectional view diagrammatically showing the motion detecting apparatus according to the third variation of the present embodiment.

FIG. 13 describes a motion detecting system according to the present embodiment.

FIG. 14 diagrammatically shows a state in which a user wears the motion detecting system according to the present embodiment.

FIG. 15 diagrammatically shows another state in which the user wears the motion detecting system according to the present embodiment.

FIG. 16 diagrammatically shows another state in which the user wears the motion detecting system according to the present embodiment.

### DESCRIPTION OF EXEMPLARY EMBODIMENTS

A preferable embodiment of the invention will be described below in detail with reference to the drawings. It is not intended that the embodiment described below unduly limits the contents of the invention set forth in the appended claims. Further, all configurations described below are not necessarily essential configuration requirements of the invention.

### 1. Motion Detecting Apparatus

The following description will be made with reference to a motion detecting apparatus that detects a physical quantity based on motion in a user's running activity (including walking activity), but the motion detecting apparatus according to the present embodiment can also be used as a motion detecting apparatus that detects a physical quantity based on motion other than running activity.

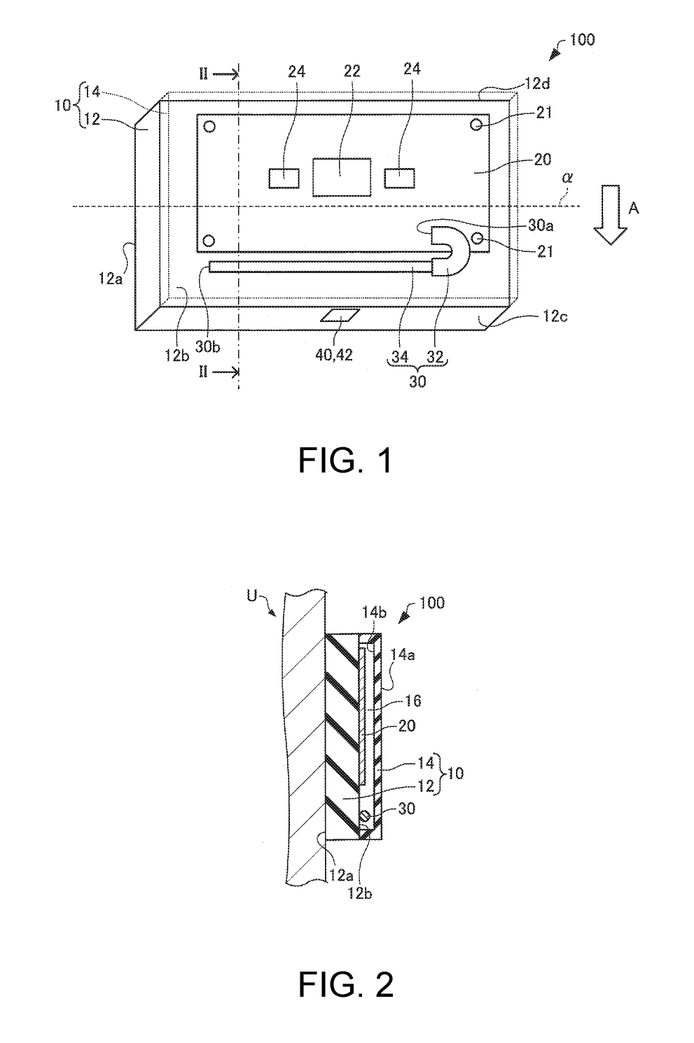

The motion detecting apparatus according to the present embodiment will first be described with reference to the drawings. FIG. 1 is a perspective view diagrammatically showing a motion detecting apparatus 100 according to the present embodiment. FIG. 2 is a cross-sectional view taken along the line II-II in FIG. 1 and diagrammatically showing the motion detecting apparatus 100 according to the present embodiment.

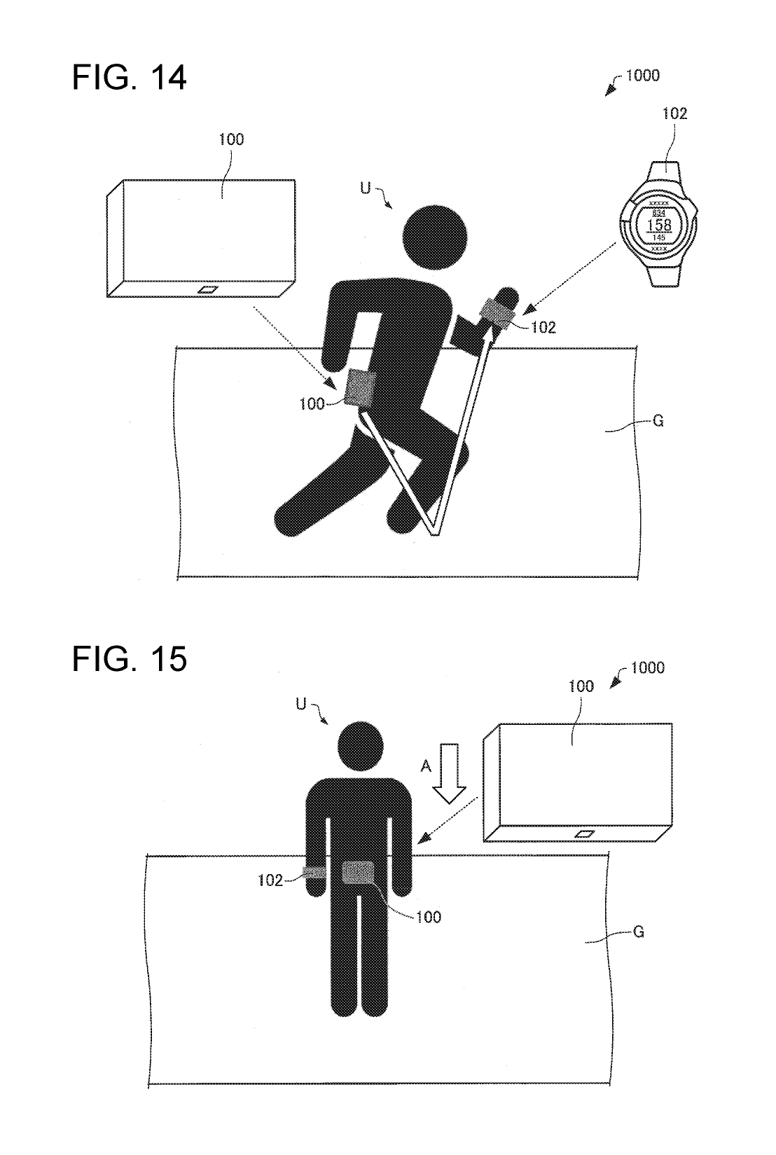

The motion detecting apparatus 100 includes a case body 10, an electronic substrate 20, an inertial sensor 22, electronic parts 24, an antenna 30, and a USB (universal serial bus) terminal 40, as shown in FIGS. 1 and 2. The motion detecting apparatus 100 is configured to be worn on a user (person) U. In other words, the user can wear the motion detecting apparatus 100. Specifically, the motion detecting apparatus 100 is configured to be worn on the waist of the user U (see FIG. 14, which will be described later). The motion detecting apparatus 100 may be worn on the waist of the user U by use of a band that is not shown.

The case body 10 accommodates the electronic substrate 20, the inertial sensor 22, the electronic parts 24, and the antenna 30. In the illustrated example, the case body 10 has a box-like shape and is a box-shaped enclosure. The case body 10 has a base 12 and a lid 14. In FIG. 1, the lid 14 is drawn as a transparent lid for convenience.

The base 12 of the case body 10 has, for example, a box-like shape. The base 12 has a surface (first surface) 12a and a surface 12b, which faces away from the surface 12a. The surface 12a is a surface that faces the user U (surface worn on user U) when the user U wears the motion detecting apparatus 100. In the example shown in FIG. 2, the surface 12a is a surface that comes into contact with the user U when the user U wears the motion detecting apparatus 100. The base 12 further has a surface 12c, in which the USB terminal 40 is disposed, and a surface 12d, which faces away from the surface 12c. The surface 12c is connected to the surfaces 12a and 12b. The surface 12d is connected to the surfaces 12a and 12b. The surfaces 12a, 12c, and 12d form the outer shape of the motion detecting apparatus 100. The base 12 is made, for example, of a plastic material or any other resin material. The case body 10 has, for example, a rectangular shape in a plan view (when viewed in direction of normal to surface 12a of base 12).

The lid 14 of the case body 10 is, for example, so shaped that a recess 16 is formed, as shown in FIG. 2, and the electronic substrate 20, the inertial sensor 22, the electronic parts 24, and the antenna 30 are accommodated in the recess 16. Although not shown, the surface 12b of the base 12 may also be provided with a recess. The lid 14 has a surface (second surface) 14a and a surface 14b, which faces away from the surface 14a. The surface 14a is a surface that faces away from the surface 12a of the base 12 and forms the outer shape of the motion detecting apparatus 100. The surface 14b is a surface that forms the bottom surface of the recess 16. The lid 14 is made, for example, of the same material as that of the base 12. The lid 14 and the base 12 may be bonded to each other with an adhesive or may be joined to each other with screws or any other fasteners.

The electronic substrate 20 is disposed on the surface 12b of the base 12. In the example shown in FIG. 1, the electronic substrate 20 is fixed to the surface 12b with screws 21. The electronic substrate 20 is, for example, a printed circuit board. The electronic substrate 20 is connected to the antenna 30.

The inertial sensor 22 is mounted on the electronic substrate 20. The inertial sensor 22 is formed, for example, of a gyro sensor (angular velocity sensor) and an acceleration sensor. Specifically, the inertial sensor 22 is formed of three gyro sensors having three axes perpendicular to one another as detection axes and three acceleration sensors having three axes perpendicular to one another as detection axes. The position of the inertial sensor 22 is not limited to a specific position.

The electronic parts 24 are disposed on the electronic substrate 20. The electronic parts 24 are, for example, disposed in a plurality of positions. Examples of the electronic parts 24 may include a memory and a CPU (central processing unit). For example, these two electronic parts are mounted on the electronic substrate 20. Although not shown, other electronic parts may be mounted on the electronic substrate 20 in addition to the memory and the CPU. The position of each of the electronic parts 24 is not limited to a specific position. The motion detecting apparatus 100 may use a result of detection performed by the inertial sensor 22 to analyze motion of the user U and generate motion analysis information that is information on a result of the analysis. The generated motion analysis information may be stored in the memory, which is one of the electronic parts 24.

One end 30a of the antenna 30 is connected to the electronic substrate 20. When the user U wears the motion detecting apparatus (when motion detecting apparatus 100 is attached to user U), at least part of the antenna 30 is located on the ground side. The term “ground side” means, for example, the side toward which the gravity is applied with the user U standing erect (see FIG. 15, which will be described later). In FIG. 1, it is assumed that the direction labeled with the arrow A is the direction of gravity. Further, the phrase “located on the ground side” means that in a plan view (viewed in direction in which base 12 and electronic substrate 20 overlap with each other, viewed in direction of normal to surface 12a of base 12), an object in question is disposed on the ground side with respect to an imaginary straight lie a that passes through the center of the surface 12b of the base 12 and is parallel to the horizontal direction (direction perpendicular to direction labeled with arrow A)). In the plan view, at least part of the antenna 30 is located in a position closer to the surface 12c of the base 12 than to the surface 12d. In the example shown in FIG. 1, the entire antenna 30 is located on the side pointed by the arrow A with respect to the imaginary straight line α when the user U wears the motion detecting apparatus 100.

In the plan view, at least part of the antenna 30 is so disposed as not to overlap with the inertial sensor 22 or the electronic parts 24. In the illustrated example, the entire antenna 30 is so disposed as not to overlap with the inertial sensor 22 or the electronic parts 24 in the plan view.

The antenna 30 has, for example, a first section 32, which forms the one end 30a of the antenna 30, and a second section 34, which forms the other end 30b of the antenna 30. In the illustrated example, the first section 32 has a curved shape and connects the electronic substrate 20 to the second section 34. The second section 34 extends from the first section 32 along the direction perpendicular to the direction labeled with the arrow A. The second section 34 has, for example, a rod-like shape. The second section 34 is disposed in a position shifted from the electronic substrate 20 in the direction labeled with the arrow A. In the illustrated example, the second section 34 is wider and longer than the first section 32. The antenna 30 is made, for example, of a metal.

The antenna 30 transmits a signal based on data outputted from the inertial sensor 22 to a notification apparatus 102 (see FIG. 14, which will be described later), which is attached to the user U. The motion detecting apparatus 100 can therefore wirelessly communicate with the notification apparatus 102. The second section 34 can, for example, transmit a greater number of signals than the first section 32. The antenna 30 is, for example, a sleeve antenna.

The USB terminal 40 is located on the ground-side surface (third surface) 12c when the user U wears the motion detecting apparatus 100. The term “ground-side surface” is a surface that faces the ground with the user U standing erect. In the example shown in FIG. 1, the USB terminal 40 is covered with a cover 42 made, for example, of a rubber material. The USB terminal 40 may instead be disposed in the ground-side surface of the lid 14.

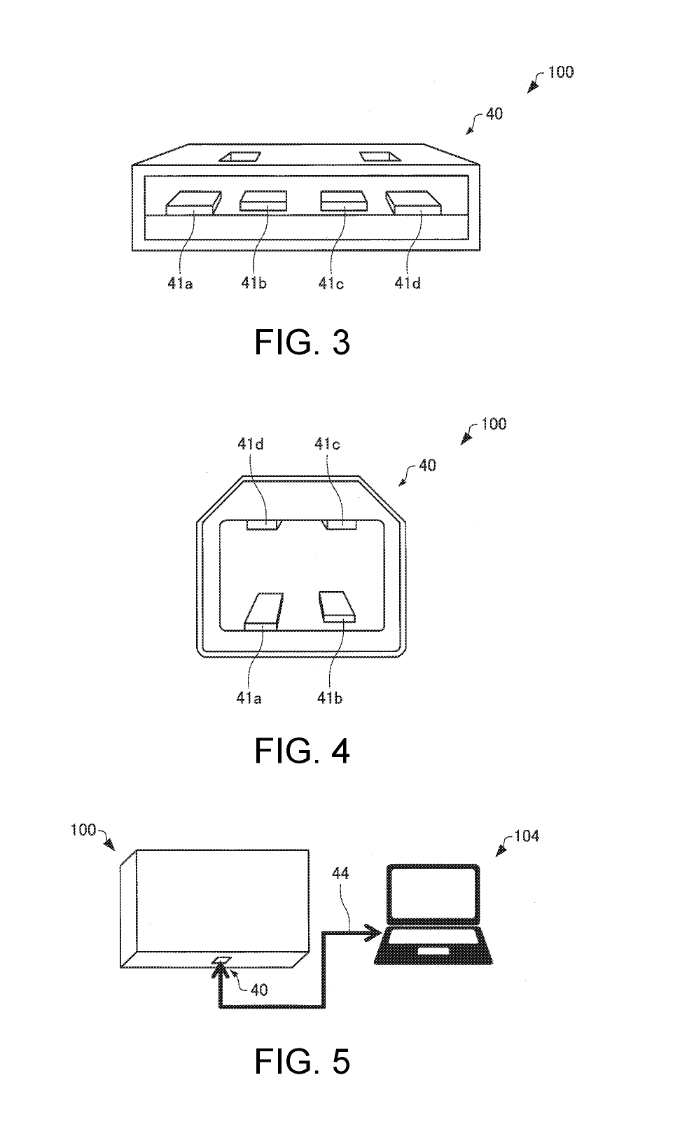

The USB terminal 40 may have a form in which four electrodes (signal lines) 41a, 41b, 41c, and 41d are arranged in line, as shown in FIG. 3. For example, the electrode 41a is a GND electrode, the electrode 41b is a D+ electrode, the electrode 41c is a D− electrode, and the electrode 41d is a V_{BUS} electrode. The USB terminal 40 may instead have a form in which the electrodes 41a and 41b are disposed side by side, the electrode 41c is so disposed as to face the electrode 41b, and the electrode 41d is so disposed as to face the electrode 41a, as shown in FIG. 4.

A USB cable (USB/serial conversion cable, for example) 44 can be inserted into the USB terminal 40 to allow the motion detecting apparatus 100 to be connected to a personal computer 104 via the USB cable, as shown in FIG. 5. The USB terminal 40 is a terminal for connecting the motion detecting apparatus 100 to an external apparatus, as described above. The motion detecting apparatus 100 includes a memory, and the memory can store the motion analysis information, as described above, but the memory of the motion detecting apparatus 100 has a small memory size. Therefore, the motion detecting apparatus 100 can be regularly connected to the personal computer 104, and the motion detection information can be extracted from the motion detecting apparatus 100 and stored in the personal computer 104.

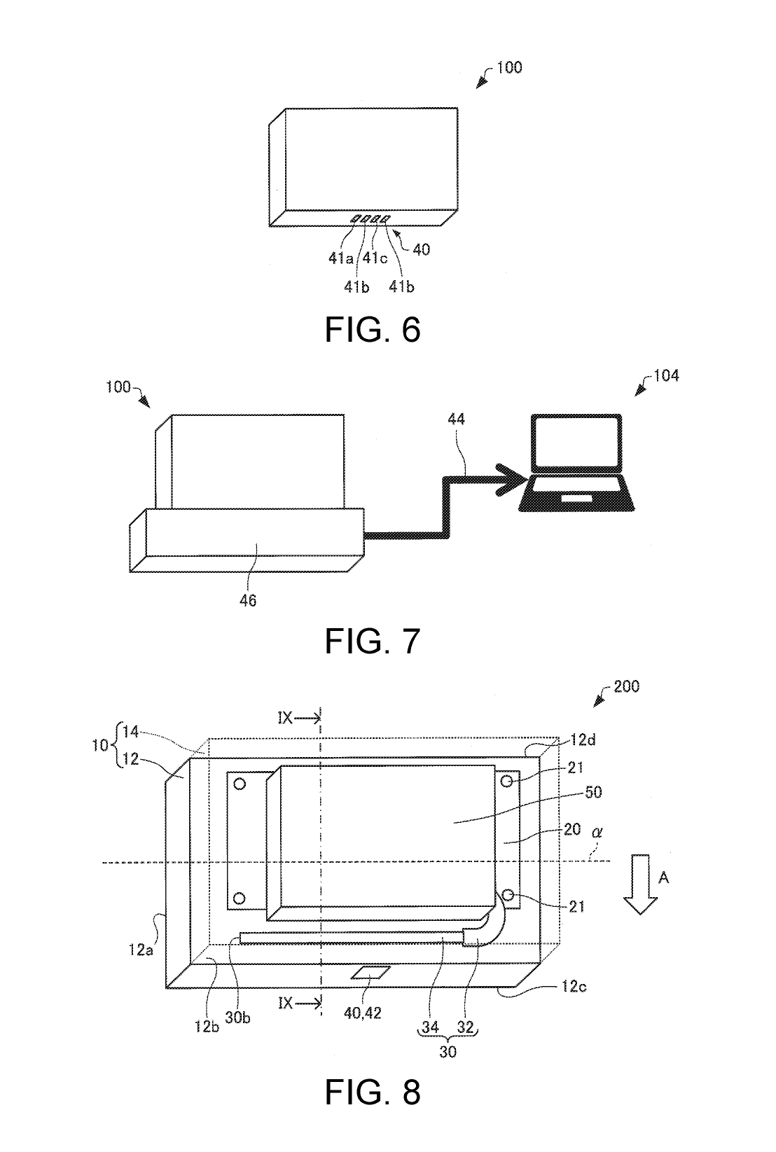

No cover that covers the USB terminal 40 may be provided so that the electrodes 41a, 41b, 41c, and 41d are exposed, as shown in FIG. 6. In this case, the electrodes 41a, 41b, 41c, and 41d may be caused to come into contact with terminals in a cradle 46, to which the USB cable 44 is connected, so that the motion detecting apparatus 100 is connected to the personal computer 104 via the cradle 46, as shown in FIG. 7.

The motion detecting apparatus 100 has, for example, the following features.

When the user U wears the motion detecting apparatus 100 (when motion detecting apparatus 100 is attached to user U), at least part of the antenna 30 is located on the ground side (side pointed by arrow A). The thus configured motion detecting apparatus 100 allows the distance between the antenna 30 and the ground to be shorter than in a case where the antenna 30 is disposed on the side opposite the side pointed by the arrow A. As a result, a signal transmitted from the antenna 30 can be more reliably reflected off the ground, whereby the motion detecting apparatus 100 can stably perform the wireless communication. The wireless communication between the motion detecting apparatus 100 and the notification apparatus 102 is performed by causing a signal from the antennal 30 to be reflected off the ground (the wireless communication will be described later in detail).

In the motion detecting apparatus 100, the antenna 30 is so disposed not to overlap with the inertial sensor 22 or the electronic parts 24 in the plan view. The thus configured motion detecting apparatus 100 allows the distance between the antenna 30 and the inertial sensor 22 and the distances between the antenna 30 and the electronic parts 24 to be greater than in a case where the antenna 30 overlaps with the inertial sensor 22 and the electronic parts 24. The motion detecting apparatus 100 can therefore suppress entry of noise into signals transmitted by the inertial sensor 22 and the electronic parts 24 via the antenna 30.

The motion detecting apparatus 100 includes the USB terminal 40 for connecting the motion detecting apparatus 100 to an external apparatus, and the USB terminal 40 is disposed in the third surface 12c, which is one of the surfaces of the case body 10 (side surfaces of enclosure) and faces the ground side when the motion detecting apparatus 100 is attached to the user U. The thus configured motion detecting apparatus 100 can suppress entry of sweat of the user U, rainwater, and other objects into the motion detecting apparatus 100 via the USB terminal 40 as compared with a case where the USB terminal 40 is disposed, for example, in the surface 12d. As a result, in the motion detecting apparatus 100, rust and other undesirable phenomena due to sweat of the user U, rainwater, and other objects is unlikely to occur. Further, the motion detecting apparatus 100, which is provided with the cover 42, can more reliably suppress entry of sweat of the user U, rainwater, and other objects into the motion detecting apparatus 100 via the USB terminal 40.

### 2. Variations of Motion Detecting Apparatus

### 2.1. First Variation

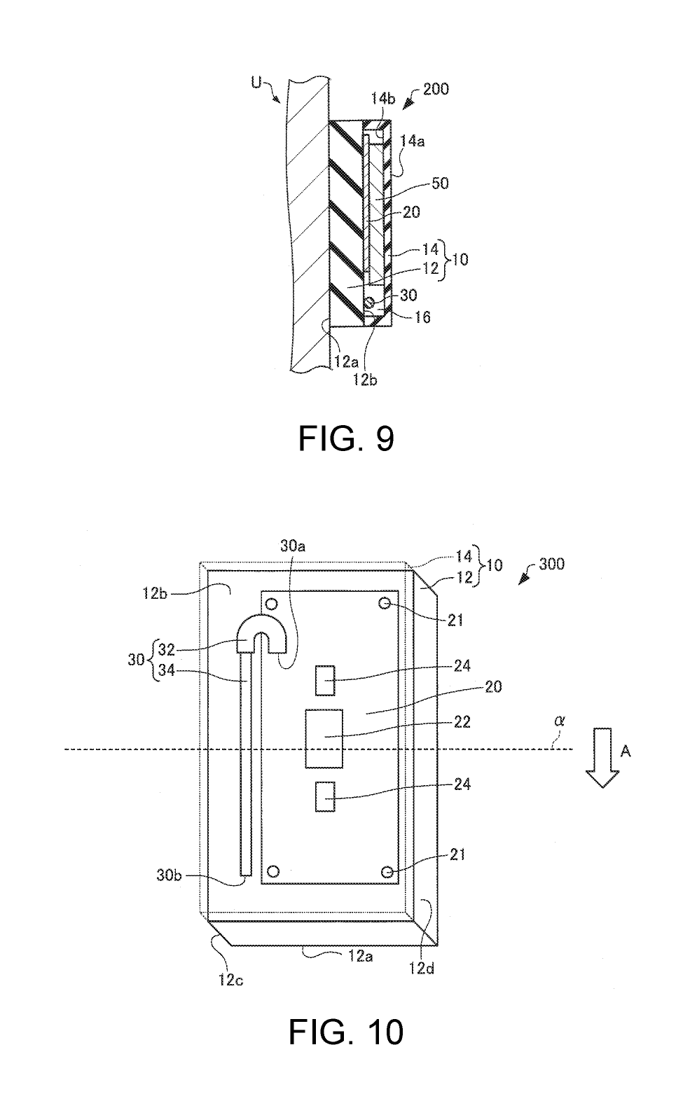

A motion detecting apparatus according to a first variation of the present embodiment will next be described with reference to the drawings. FIG. 8 is a perspective view diagrammatically showing a motion detecting apparatus 200 according to the first variation of the present embodiment. FIG. 9 is a cross-sectional view taken along the line IX-IX in FIG. 8 and diagrammatically showing the motion detecting apparatus 200 according to the first variation of the present embodiment. In FIG. 8, the lid 14 is drawn as a transparent lid for convenience.

In the following description of the motion detecting apparatus 200 according to the first variation of the present embodiment, members having the same functions as those of the members that from the motion detecting apparatus 100 according to the present embodiment have the same reference characters and will not be described in detail. The same holds true for motion detecting apparatus according to second and third variations of the present embodiment, which will be described later.

The motion detecting apparatus 200 differs from the motion detecting apparatus 100 described above in that the motion detecting apparatus 200 includes a battery 50, as shown in FIGS. 8 and 9.

The battery 50 is accommodated in the case body 10. The battery 50 is located on the side facing the second surface 14a (side opposite the side facing the user U to which the motion detecting apparatus is attached), which is opposite the first surface 12a, which faces the user U, when the user U wears the motion detecting apparatus 200 (when motion detecting apparatus 200 is attached to user). That is, the distance between the battery 50 and the first surface 12a is greater than the distance between the battery 50 and the second surface 14a. In the example shown in FIG. 9, the battery 50 is disposed on the surface 14b of the lid 14. The battery 50 is fixed to the surface 14b, for example, with an adhesive or a double-sided adhesive tape. The battery 50 can, for example, supply the electronic substrate 20 and other components with electric power.

The electronic substrate 20 is disposed between the first surface 12a and the battery 50. The electronic substrate 20 is disposed between the user U and the battery 50 when the user U wears the motion detecting apparatus 200. The second section 34 of the antenna 30 does not overlap with the battery 50 in the plan view. In the example shown in FIG. 8, the second section 34 is disposed in a position shifted from the battery 50 toward the side pointed with the arrow A.

The motion detecting apparatus 200 has, for example, the following features.

The motion detecting apparatus 200 can stably perform the wireless communication, as the motion detecting apparatus 100 can.

In the motion detecting apparatus 200, the battery 50 is disposed on the side facing the second surface 14a, which is opposite the first surface 12a, which faces the user U. Therefore, when the user U wears the motion detecting apparatus 200, the distance between the user U and the battery 50 can be greater than in a case where the battery 50 is disposed on the side facing the first surface 12a. The influence of heat that the user U receives from the battery 50 can therefore be reduced. The motion detecting apparatus 200 can therefore improve the safety of the user U. Since the battery 50 dissipates heat, for example, immediately after it is charged, the heat from the battery 50 is likely to affect the user U when the user U wears the motion detecting apparatus 200 for a long period, but the motion detecting apparatus 200 can be reduce the influence of the heat, as described above.

In the motion detecting apparatus 200, the electronic substrate 20 is located between the first surface 12a, which faces the user U, and the battery 50 when the user U wears the motion detecting apparatus 200. Therefore, even if the battery 50 dissipates heat, the electronic substrate in the motion detecting apparatus 200 can suppress transmission of the heat to the user U. As a result, the motion detecting apparatus 200 can further reduce the influence of the heat that the user U receives from the battery 50.

### 2.2. Second Variation

A motion detecting apparatus according to a second variation of the present embodiment will next be described with reference to the drawings. FIG. 10 is a perspective view diagrammatically showing a motion detecting apparatus 300 according to the second variation of the present embodiment. In FIG. 10, the lid 14 is drawn as a transparent lid for convenience.

In the motion detecting apparatus 100 described above, the entire antenna 30 is located on the side pointed by the arrow A with respect to the imaginary straight line α when the user U wears the motion detecting apparatus 100, as shown in FIG. 1.

In contrast, in the motion detecting apparatus 300, part of the antenna 30 is located on the side pointed by the arrow A with respect to the imaginary straight line α when the user U wears the motion detecting apparatus 300, as shown in FIG. 10. Specifically, the one end 30a of the antenna 30 is located on the side opposite the side pointed with the arrow A with respect to the imaginary straight line α, and the other end 30b of the antenna 30 is located on the side pointed by the arrow A with respect to the imaginary straight line α. The other end 30b of the antenna 30 faces the side pointed with the arrow A. That is, the other end 30b of the antenna 30 is so located as to face the ground when the user U wears the motion detecting apparatus 300.

The motion detecting apparatus 300 can stably perform the wireless communication, as the motion detecting apparatus 100 can.

In the motion detecting apparatus 300, the other end 30b of the antenna 30 is so located as to face the ground when the user U wears the motion detecting apparatus 300. Therefore, in the motion detecting apparatus 300, a signal transmitted from the antenna 30 can be readily radiated toward the ground, whereby the signal transmitted from the antenna 30 can be more reliably reflected off the ground.

Although not shown, the motion detecting apparatus 300 may include the battery 50, as the motion detecting apparatus 200 does.

### 2.3. Third Variation

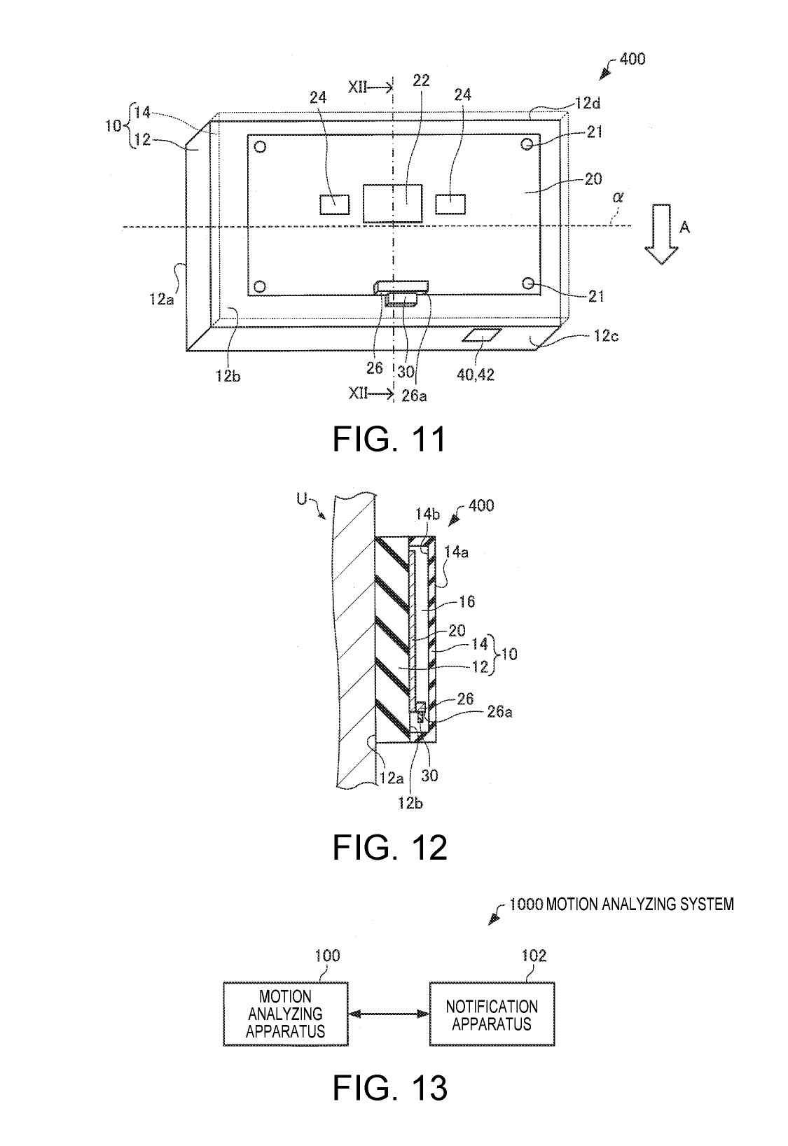

A motion detecting apparatus according to a third variation of the present embodiment will next be described with reference to the drawings. FIG. 11 is a perspective view diagrammatically showing a motion detecting apparatus 400 according to the third variation of the present embodiment. FIG. 12 is a cross-sectional view taken along the line XII-XII in FIG. 11 and diagrammatically showing the motion detecting apparatus 400 according to the third variation of the present embodiment. In FIG. 11, the lid 14 is drawn as a transparent lid for convenience. Further, in FIG. 12, the inertial sensor 22 is not shown.

In the motion detecting apparatus 100 described above, the antenna 30 is a sleeve antenna, as shown in FIGS. 1 and 2.

In contrast, in the motion detecting apparatus 400, the antenna 30 is a patch antenna, as shown in FIGS. 11 and 12. In the motion detecting apparatus 400, the electronic substrate 20 has a protrusion 26, which protrudes toward the lid 14. The antenna 30 is disposed on a surface 26a of the protrusion 26. The surface 26a is a surface that faces the side pointed with the arrow A (ground-side surface). The USB terminal 40 is, for example, not disposed in a position shifted from the antenna 30 in the direction labeled with the arrow A but is disposed in a position shifted from the antenna 30 in a direction different from the direction labeled with the arrow A in the plan view.

In the motion detecting apparatus 400, the antenna 30 is a patch antenna. A patch antenna, for example, has higher sensitivity in a single direction than a sleeve antenna and can therefore restrict the radiation direction and emit no electric wave in an unnecessary direction. Therefore, in the motion detecting apparatus 400, a signal transmitted from the antenna 30 is allowed to be reflected off the ground more reliably than in the case where a sleeve antenna is used, whereby the wireless communication can be stably performed.

Although not shown, the motion detecting apparatus 400 may include the battery 50, as the motion detecting apparatus 200 does.

### 3. Motion Detecting System

A motion detecting system according to the present embodiment will next be described with reference to the drawings. FIG. 13 describes a motion detecting system 1000 according to the present embodiment. FIGS. 14 and 15 each diagrammatically show a state in which the user U wears the motion detecting system 1000 according to the present embodiment. FIG. 14 shows a state in which the user U is running, and FIG. 15 shows a state in which the user U stands erect.

The motion detecting system according to the present embodiment of the invention includes any of the motion detecting apparatus according to the present embodiment of the invention. In the following sections, the motion detecting system 1000 including the motion detecting apparatus 100 as the motion detecting apparatus according to the present embodiment of the invention will be described.

The motion detecting system 1000 includes the motion detecting apparatus 100 and the notification apparatus 102, as shown in FIGS. 13 to 15.

The motion detecting apparatus 100 is configured to be worn on the waist of the user U, as shown in FIGS. 14 and 15. In the illustrated example, the motion detecting apparatus 100 is worn on a central portion of the waist but may instead be worn on a right portion of the waist or a left portion of the waist. When the user U wears the motion detecting apparatus 100, as shown in FIG. 15, at least part of the antenna 30 (see FIG. 1) is located on the side facing the ground G. When the user U wears the motion detecting apparatus 100 as shown in FIG. 15, at least part of the antenna 30 is located on the side opposite the head of the user U. The motion detecting apparatus 100 is, for example, an apparatus that analyzes the motion of the user U who is running.

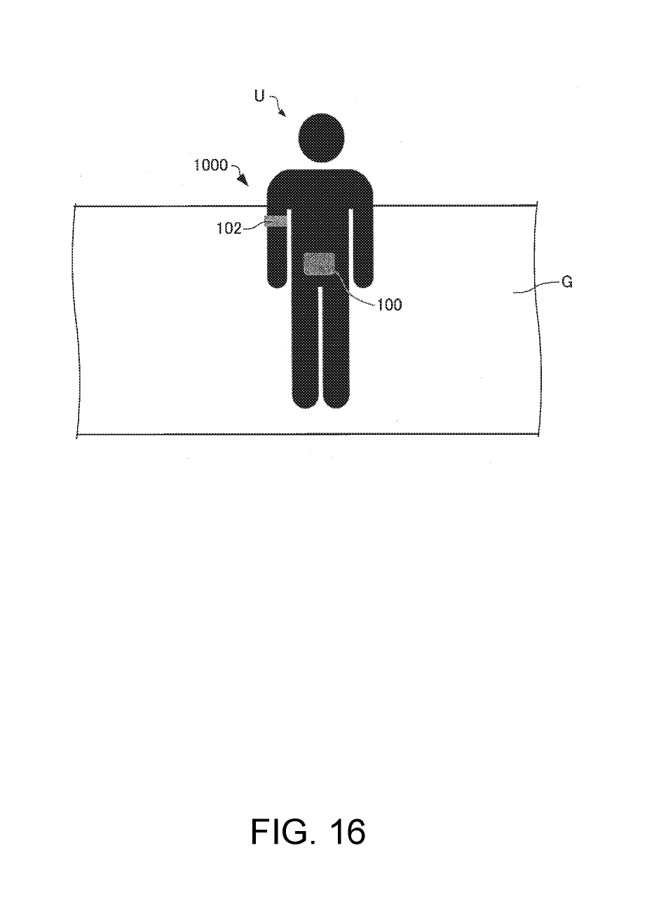

The notification apparatus 102 is configured to be worn around an arm of the user U. The notification apparatus 102 is a wrist-type (wristwatch-type) portable information instrument and worn around a wrist of the user U (wrist of left arm in illustrated example). The notification apparatus 102 may be a smartphone or any other portable information instrument, or the notification apparatus 102 may be accommodated in a case, and the case may be worn on an upper arm (portion from shoulder to elbow) of the user U, as shown in FIG. 16. The notification apparatus 102 is an apparatus that notifies the user U of the state of the running motion and a result of the running.

The user U operates the notification apparatus 102 when the user U starts running to instruct the motion detecting apparatus 100 to start measurement and operates the notification apparatus 102 when the user U stops running to instruct the motion detecting apparatus 100 to stop the measurement. The notification apparatus 102 transmits a command that instructs the start of the measurement and a command that instructs the end of the measurement to the motion detecting apparatus 100 in accordance with operation of the user U.

The motion detecting apparatus 100, when it receives, for example, the measurement start command, causes the inertial sensor 22 to start measurement, uses a result of the measurement to calculate values of a variety of motion indices that are indices relating to the running performance (example of motion performance) of the user U, and generates motion analysis information containing the values of the variety of motion indices as information on a result of the analysis of the user's running motion. The motion detecting apparatus 100 transmits a signal containing the generated motion analysis information to the notification apparatus 102. The notification apparatus 102 receives the signal containing the motion analysis information from the motion detecting apparatus 100, compares the values of the variety of motion indices, for example, with target values set in advance, and notifies the user of how good or bad each of the motion indices is primarily in the form of sound or vibration. The user U can therefore run while recognizing how good or bad each of the motion indices is.

The variety of motion indices calculated in the motion detecting apparatus 100 are not limited to specific ones and may, for example, include the amount of braking at the time of landing (motion index defined as the amount of speed reduced by landing), the degree of directly-under-body landing (motion index representing if user has successfully landed a point directly under the body), the driving force (motion index defined as the amount of speed having increased in traveling direction by action of kicking ground), the forward tilting angle (motion index representing how much torso of user U inclines with respect to ground), and the stream of leg motion (motion index representing how much rearward kicking leg is when kicking leg then lands).

The motion detecting apparatus 100, when it receives, for example, the measurement stop command, causes the inertial sensor 22 to stop the measurement, generates information on a result of the running of the user U (running result information: travel distance and running speed), and transmits the information to the notification apparatus 102. The notification apparatus 102 receives the running result information from the motion detecting apparatus 100 and notifies the user U of the running result information in the form of letters or an image. The user U can therefore recognize the running result information immediately after the completion of the running.

The data communication between the motion detecting apparatus 100 and the notification apparatus 102 is performed over wireless communication. Specifically, the wireless communication between the motion detecting apparatus 100 and the notification apparatus 102 is performed in compliance with Bluetooth (registered trademark) or Wi-Fi (wireless fidelity). The motion detecting system 1000 may include, for example, the personal computer 104 (see FIG. 5).

The wireless communication between the motion detecting apparatus 100 and the notification apparatus 102 is performed via the portion with which each foot (shoe) of the user U comes into contact during running. For example, the motion detecting apparatus 100 transmits a signal containing the motion analysis information from the antenna 30 to the notification apparatus 102 via the portion with which each foot (shoe) of the user U comes into contact during running. That is, the signal transmitted from the antenna 30 is radiated toward the ground G, reflected off the ground G, and received by the notification apparatus 102, whereby the motion detecting apparatus 100 can wirelessly communicate with the notification apparatus 102. The phrase “portion with which each foot (shoe) of the user U comes into contact during running” is the ground G in the example shown in FIG. 14 but is not limited to a specific portion and may instead be a road surface, such as an asphalt-paving road surface, a floor of a gymnasium, or any other surface.

The motion detecting system 1000 includes the motion detecting apparatus 100. Therefore, for example, even when the motion detecting apparatus 100 is worn on the waist of the user U, and the notification apparatus 102 is worn on an arm of the user U, so that the body of the user U is present between the motion detecting apparatus 100 and the notification apparatus 102, a signal transmitted from the antenna 30 is radiated toward the ground G, reflected off the ground G, and received by the notification apparatus 102. The motion detecting system 1000 can therefore stably perform the wireless communication between the motion detecting apparatus 100 and the notification apparatus 102. As a result, the motion detecting system 1000 allows, for example, improvement in the quality of the wireless communication.

The above example has been described with reference to the case where the motion detecting apparatus 100 uses a result of the detection performed by the inertial sensor 22 (output data, measured value detected by inertial sensor 22) to analyze motion of the user U, generates motion analysis information that is information on a result of the analysis, and transmits a signal containing the motion analysis information (signal based on output data) to the notification apparatus 102. Instead, in the motion detecting system according to the embodiment of the invention, the motion detecting apparatus 100 may transmit data outputted from the inertial sensor 22 as a signal based on the output data to the notification apparatus 102, and the notification apparatus 200 may analyze the user's motion on the basis of the output data and generate the motion analysis information. That is, the antenna 30 may transmit data outputted from the inertial sensor 22 to the notification apparatus 102 or may transmit a signal containing the motion analysis information to the notification apparatus 102.

In the invention, part of the configuration descried in the present application may be omitted or the embodiment and the variations may be combined with each other to the extent that the resultant embodiment provides the features and effects descried in the present application.

The invention encompasses substantially the same configuration as the configuration described in the embodiment (for example, a configuration having the same function, using the same method, and providing the same result or a configuration having the same purpose and providing the same effect). Further, the invention encompasses a configuration in which an inessential portion of the configuration described in the embodiment is replaced. Moreover, the invention encompasses a configuration that provides the same advantageous effect as that provided by the configuration described in the embodiment or a configuration that can achieve the same purpose as that achieved by the configuration described in the embodiment. Further, the invention encompasses a configuration in which a known technology is added to the configuration described in the embodiment.

The entire disclosure of Japanese Patent Application No. 2016-173420 filed Sep. 6, 2016 is expressly incorporated by reference herein.

What is claimed is:

1. A motion detecting apparatus comprising:
an inertial sensor; and
an antenna via which a signal based on data outputted from the inertial sensor is transmitted,
wherein when the motion detecting apparatus is attached to a user, at least part of the antenna is located on a ground side as compared with the inertial sensor.

2. The motion detecting apparatus according to claim 1,
further comprising an electronic substrate connected to the antenna.

3. The motion detecting apparatus according to claim 2,
wherein the inertial sensor and an electronic part are mounted on the electronic substrate, and
in a plan view viewed in a direction that intersects a first surface that is worn on the user,
at least part of the antenna is so located as not to overlap with the electronic part or the inertial sensor.

4. The motion detecting apparatus according to claim 1,
further comprising a box-shaped enclosure that accommodates a battery,
wherein when the motion detecting apparatus is attached to the user, the battery is located on a side opposite a side facing the user's site to which the motion detecting apparatus is attached.

5. The motion detecting apparatus according to claim 4,
wherein the electronic substrate is disposed between a first surface and the battery.

6. The motion detecting apparatus according to claim 4,
further comprising a terminal connected to another instrument,
wherein when the motion detecting apparatus is attached to the user, the terminal is located on a ground-side side surface out of side surfaces of the enclosure.

7. The motion detecting apparatus according to claim 1,
wherein the motion detecting apparatus is worn on the user's waist.

8. A motion detecting system comprising:
the motion detecting apparatus according to claim 1; and
a notification apparatus.

9. A motion detecting system comprising:
the motion detecting apparatus according to claim 2; and
a notification apparatus.

10. A motion detecting system comprising:
the motion detecting apparatus according to claim 3; and
a notification apparatus.

11. A motion detecting system comprising:
the motion detecting apparatus according to claim 4; and
a notification apparatus.

12. A motion detecting system comprising:
the motion detecting apparatus according to claim 5; and
a notification apparatus.

13. A motion detecting system comprising:
the motion detecting apparatus according to claim 6; and
a notification apparatus.

14. A motion detecting system comprising:
the motion detecting apparatus according to claim 7; and
a notification apparatus.

15. The motion detecting system according to claim 8,
wherein the notification apparatus is worn on the user's arm.

16. A method for attaching, to a user, a motion detecting apparatus including an inertial sensor and an antenna via which a signal based on data outputted from the inertial sensor is transmitted, the method comprising
attaching the motion detecting apparatus in such a way that the antenna is located in a position shifted from the inertial sensor toward a ground.
